(19) United States
(12) Patent Application Publication
(10) Pub. No.: US 2025/0256972 A1
(43) Pub. Date: Aug. 14, 2025

(54) NANOSTRUCTURE, NANOCOMPOSITE, AND IMPLEMENTATIONS THEREOF

(71) Applicant: Theranautilus Pvt. Ltd., Karnataka Bangalore (IN)

(72) Inventors: Debayan DASGUPTA, Karnataka Bangalore (IN); Shanmukh Srinivas PEDDI, Karnataka Bangalore (IN); Prajwal Bramhavar HEGDE, Karnataka Bangalore (IN); Ambarish GHOSH, Karnataka Bangalore (IN)

(21) Appl. No.: 18/857,982

(22) PCT Filed: Apr. 20, 2023
(86) PCT No.: PCT/IN2023/050384

§ 371 Date: Oct. 18, 2024

(30) Foreign Application Priority Data
Apr. 20, 2022 (IN) 202241023397

(51) Int. Cl. C01B 33/24; A61K 9/06; A61K 33/06; A61K 33/26; A61K 47/02; B82Y 25/00; B82Y 30/00; C01G 49/08

(52) U.S. Cl. CPC C01B 33/24; A61K 9/06; A61K 33/06; A61K 33/26; A61K 47/02; C01G 49/08; B82Y 25/00; B82Y 30/00; C01P 2004/62; C01P 2004/64; C01P 2004/84; C01P 2006/12; C01P 2006/16; C01P 2006/42

(57) ABSTRACT

The present disclosure relates to a nanostructure containing: 50 to 80% (w/w) of a magnetic material; and 20 to 50% (w/w) of calcium silicate. The present disclosure further relates to a nanocomposite containing the nanostructure as disclosed herein with an additive. The present disclosure also provides a gel containing the nanostructure or the nanocomposite and additives, and methods thereof.

### FIELD OF INVENTION

The subject matter disclosed herein broadly relates to the field of oral healthcare, and in particular relates to magnetic nanostructures and nanocomposites for use in the treatment of dental hypersensitivity.

### BACKGROUND OF THE INVENTION

Dentin hypersensitivity (DH) as a chronic disease is increasingly prevalent among adults, which consists of sharp pain arising from exposed dentin in response to a varied assortment of stimuli, for example, dietary factors, such as an ice-cold beverage, to even environmental considerations, such as the exposure to atmospheric air on a cold winter's day. The heterogeneity of problems associated with DH ranges from minor inconvenience to the patient, to a near incapacitating quality-of-life disturbance. It can affect patients of any age group and most commonly affects the canines and premolars of both the arches. The intensity and degree of sensitivity depend on different factors and are different in different people.

The major causal factors of DH include the loss of hard tissue which can result in attrition, abrasion, erosion and bruxism, and gingival recession which can be caused by periodontal disease, bad brushing, and periodontal interventions. Microscopic studies have revealed that said factors accentuate the DH in places of exposed dentine due to removal or deterioration of the covering layer of enamel. The hydrodynamic theory proposed by Brannstorm is based on the movement of the fluid inside the dentinal tubules which are open between dentine surfaces and are exposed to the environment and pulp. As a result of any stimuli in the close vicinity of the exposed dentine surface, the movement of fluid in the dentinal tubules gets affected, either towards the pulp or away from the pulp. Said change in the movement of dentinal fluid kicks in the signals in the nerve tissues in the pulp and results in a feeling of pain.

Emerging research on determining etiologic factors in the causation of the disease, its diagnosis and its treatment have uncovered many materials and methods in order to reduce or remove sensitivity. Such materials usually exert their effects through “sealing dentinal tubules” or through “disturbing the transmission of nerve impulses”. Some of these materials include the use of toothpastes containing potassium salts, fluoride composites, resins, laser; bioglass, and so on. For instance, US20140127142A1 relates to a toothpaste composition for DH that effectively prevents pain attributed to DH, instantaneously and sufficiently seals the openings of dentinal tubules in an exposed dentinal surface. Said toothpaste composition comprises, a powder having a sparingly water-soluble property; a desensitizing agent; and water.

JP5816352B2 relates to a DH inhibitor in which a liquid or water-based paste is mainly composed of tetracalcium phosphate particles (A), phosphoric acid alkali metal salt (B), acidic calcium phosphate particles (C), and water. Said inhibitor composition is used to seal dentinal tubules by rubbing them into the dentin surface.

Notwithstanding the ongoing research and already established methods, the problem of DH is evidently increasing in different age groups. Further, the failure of conventional medicaments and methods to seal the dentinal tubule opening for a longer period of time is leading to consistent problems. Due to aforesaid reasons, disturbing the transmission of nerve impulses from the exposed dentine surface to nerve tissues (pulp) seems to be a more appropriate and better solution for the prevention or eradication of DH. Thus, there is still a dire need in the state of art to develop methods or medicaments based on disrupting the transmission of nerve impulses, thereby facilitating more permanent and longtime prevention of pain caused by DH.

### SUMMARY OF THE INVENTION

In first aspect of the present disclosure, a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm.

In second aspect of the present disclosure, there is provided a nanocomposite comprising: a) 95 to 99.9% (w/w) of the nanostructure comprising (i) 50 to 80% (w/w) of a magnetic material and (ii) 20 to 50% (w/w) of calcium silicate; and b) 0.1 to 5% (w/w) of an additive selected from calcium oxide, phosphorus, sodium, strontium, phosphates, fluorine, or combinations thereof, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm.

In third aspect of the present disclosure, there is provided a process for preparing the nanostructure, the process comprising: (a) mixing an oxidizing agent, and a magnetic metal salt in a first solvent followed by addition of a base and a silicate precursor to obtain a first mixture; (b) contacting a calcium salt with the first mixture in the presence of a second solvent to obtain a second mixture; and (c) annealing the second mixture at a temperature in a range of 550 to 650° C. to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm

In fourth aspect of the present disclosure, there is provided a process of preparing the nanocomposite, the process comprising: a) preparing the nanostructure as disclosed herein; and b) immersing the nanostructure in a solution comprising a compound selected from calcium oxide, strontium salt, calcium fluoride, sodium fluoride, phosphates, or combinations thereof, at a temperature in a range of 20 to 80° C. to obtain the nanocomposite.

In fifth aspect of the present disclosure, there is provided a gel comprising: a) 0.1 to 10% (w/w) of the nanostructure or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; and g) optionally 1 to 5% (w/w) of a stabilizer.

In sixth aspect of the present disclosure, there is provided a process for preparing the gel, the process comprising: a) mixing the nanostructure or the nanocomposite as disclosed herein with a hydrogel base and a swelling agent to obtain a first solution; b) adding a pH modifier and calcium oxide powder to the first solution to obtain a second solution; and c) adding a humectant and optionally a stabilizer to obtain the gel.

In seventh aspect of the present disclosure, there is provided a method of treating dental hypersensitivity, the method comprising administering the nanostructure or the nanocomposite or the gel as disclosed herein to a subject suffering from dental hypersensitivity.

In eighth aspect of the present disclosure, there is provided a method of administering the nanostructure or the nanocomposite or the gel as disclosed herein, the method comprising applying, driving, and positioning the nanostructure or the nanocomposite or the gel as disclosed herein to an infected dentinal tubule using a magnetic cap.

In ninth aspect of the present disclosure, there is provided a device comprising the nanostructure or the nanocomposite or the gel as disclosed herein and a magnetic cap.

In tenth aspect of the present disclosure, there is provided a use of the nanostructure or the nanocomposite or the gel as disclosed herein.

These and other features, aspects, and advantages of the present subject matter will be better understood with reference to the following description. This summary is provided to introduce a selection of concepts in a simplified form. This summary is not intended to identify key features or essential features of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the high-angle annular dark-field imaging (HAADF) of the nanostructure, in accordance with an embodiment of the present disclosure.

FIG. 2 depicts the scanning electron microscopic analysis and high-angle annular dark-field imaging (HAADF), in accordance with an embodiment of the present disclosure.

FIG. 3 depicts the energy dispersive x-ray analysis of the nanostructure, in accordance with an embodiment of the present disclosure.

FIG. 4 depicts the microscopic image of a dentinal tube blocked by the nanostructures, in accordance with an embodiment of the present disclosure.

FIG. 5 depicts the microscopic image of a dentinal tube blocked by the cementing blocks formed by the nanocomposite, in accordance with an embodiment of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” used herein refers to one or more and thus includes individual components as well as mixtures/combinations.

The term “metal hydroxide” refers to chemical compounds containing a metal cation and —OH anion and are often called hydroxides of metals. For the purpose of present disclosure, metal hydroxide includes but not limited to sodium hydroxide, potassium hydroxide, calcium hydroxide, and barium hydroxide.

The term “antibacterial agent” refers to any synthetic or natural compounds which inhibit or prevent the growth and division of bacteria in an oral site. Various examples used as antibacterial agents for the purpose of the present disclosure include but not limited to hydrogen peroxide, ethanol, ciprofloxacin, norfloxacin, and gatifloxacin.

The term “magnetic cap” relates to a magnetic device comprising of a simple magnet or electromagnetic coil or other magnetic material meant for use in driving the nanostructures inside the dentine tubules. Said magnetic cap may also be termed as a magnetic clip which facilitates the movement of nanostructures (having magnetic core) in a controlled way under a magnetic field, which can be selected from the group of rotating magnetic field, an oscillating magnetic field, a gradient magnetic field, an elliptical magnetic field, a constant magnetic field, or combinations thereof.

The term “zeta potential” refers to the electrokinetic potential in colloidal systems which is used as a standard characterization technique to evaluate nanoparticle surfaces. In the present context, the zeta potential was used to assess the nanostructure's surface charge, i.e., cationic, anionic, or neutral character. The nanostructure as disclosed herein has a zeta potential in a range of −20 to −40 mV.

The term “magnetic metal salt” refers to the metal salts which exhibit magnetic strength and ability to respond to the applied magnetic field or change in the applied magnetic field. For the purpose of the present disclosure, salts selected from but not limited to iron chloride, iron nitrate, nickel chloride, nickel nitrate, cobalt chloride, cobalt nitrate, or combinations thereof, could be used as magnetic metal salts.

The term “hydrogel base” relates to a hydrating base in a gel composition favourable for use in an oral setting. For the purpose of the present disclosure, the hydrogel base may be selected from but not limited to polyacrylic acid, diutan gum, alkyl acrylate cross polymer, poloxamers, or combinations thereof. Alkyl acrylate cross polymer refers to co-polymer of various alkyl acrylates. The alkyl acrylates crosspolymers are obtained by polymerizing C₁₀₋₃₀ alkyl acrylates with various other acrylates including acrylic acid and methacrylic acid. Poloxamers are triblock copolymers which are non-ionic and comprises a hydrophobic chain of polyoxypropylene flanked by two hydrophilic chains of polyoxyethylene.

The term “pH modifier” relates to the compounds used to maintain the stability, preservability and pH of the nanostructure or nanocomposite-based gel composition as disclosed herein. The pH modifier in the context of the present disclosure, may be selected from but not limited to triethanolamine, di-sodium tetraborate, TrisBase, or combinations thereof.

The term “humectant” relates to the hygroscopic substances used to maintain moisture in the gel composition as disclosed herein. It effectively helps in extended preservation of the gel composition. Humectants, for the purpose of the present disclosure, may be selected from but not limited to glycerin, lecithin, propylene glycol, water or combinations thereof.

The term “swelling agent” relates to the substances used for creating the mesoporous or aerated texture in the gel composition. For instance, ethanol, water, or combinations thereof, may be used as swelling agent for the purpose of preparing the gel composition as disclosed herein.

The term “stabilizer” relates to the substances used to provide uniformity and stability to the gel as disclosed herein. It imparts various properties to the gel composition which helps it to be easily pumped or squeezed based on improved consistency. In the present disclosure, the stabilizer may be selected from but not limited to xanthan gum, gelatin, starch, agar glycerides, or combinations thereof.

The term “additive” as disclosed in the present disclosure includes but not limited to calcium oxide, phosphorus, sodium, strontium, phosphates, fluorine, or combinations thereof. The additive could be selected from compounds of sodium, compounds of strontium, phosphorus compounds, fluorine compounds, metal phosphates or combinations thereof. The term “strontium salt” refers to any strontium compound that could be a source for strontium and is not limited to halides, nitrates, and phosphates of strontium. The term “phosphates” includes but not limited to metal phosphates.

The term “storage modulus” refers to a mechanical property of the gel as disclosed herein that measures the stiffness of the gel. In other words, it corresponds to the gel strength which reflects the measure of the rigidity of gel composition. The storage modulus of the disclosed gel composition lies in a range of but not limited to 1 to 1000 Pa.

The term “viscosity” of the gel as disclosed herein corresponds to a measure of its resistance to deformation at a given rate. It corresponds to the “thickness” of the gel, which shall be in the range of but not limited to 1 to 10⁵ cP.

The term “piezo generator” refers to a power generation device that works on the principle of piezoelectricity and it holds the ability to convert mechanical energy. For example, vibrations in the piezoelectric materials are converted to electric energy. In the device as disclosed herein, the piezo generator generates acoustic vibrations to induce shear thinning of the nanostructure or the nanocomposite or the gel.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As it is discussed in the background, the DH has become a more prevalent disorder and can lead to both physical and psychological problems for the patient. Furthermore, it can have a negative effect on the quality of a person's life, especially with regards to dietary selection, maintaining optimal dental hygiene, and beauty aspects.

Majority of the conventional medicaments or methods developed for the prevention or inhibition of DH are based on sealing the exposed dentinal tubules, which always have been a short-term treatment approach. In such cases, as soon as the covering or sealant gets off from the sealed tubules, the DH reoccurs. Although, there are more advanced treatment strategies available in the field of art, which are based on the nerve impulse disruption approach. But still there is a need for a treatment strategy against DH which can be relied upon.

The present disclosure relates to a nanostructure, a nanocomposite, a gel composition, an oral healthcare device, and process of preparing thereof respectively. Unlike conventional medications which aim at temporarily sealing the site of sensitivity, the nanostructures as disclosed herein serve as more of a permanent solution to the problem of DH. The nanostructures as disclosed herein navigate into the dentinal tubules and block the passage, which bars the nerve impulse signals to travel from the site of sensitivity to the pulp, thereby preventing the occurrence of DH/pain. Furthermore, calcium in the nanostructures triggers bone growth in these exposed dentine thus forming a more permanent blockage. The strontium doping would desensitize the nerve ending thereby substantially reducing pain. The nanostructures are further combined with an additive such as calcium oxide along with calcium silicate, which provides the ability to form cementing blocks in the tubules. The nanostructures as disclosed herein also have the regenerative capabilities causing repair of the mineralized tissues through triggering regenerative cell signaling processes. Along with their regenerative ability, they can be remotely homed towards the affected areas through generating directive electromagnetic fields from a hand-held device due to their innate magnetic nature. With these attributes, said nanostructures can be purposed for regeneration and repair of mineralized tissues such as bones, ligaments, and teeth.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm. In another embodiment of the present disclosure, there is provided a nanostructure comprising: 60 to 70% (w/w) of a magnetic material and 25 to 40% (w/w) of calcium silicate, wherein the magnetic particle forms a core of the nanostructure and calcium silicate forms a shell of the nanostructure; the nanostructure has particle size in a range of 100 to 450 nm; surface area in a range of 1 μm² to 100 μm²; and pore size in a range of 4 nm to 18 nm.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the nanostructure encompass shape selected from sphere, ellipsoids, dumb-bell shaped linked spheres, multiple linked spheres, chains, rods, helices, or combinations thereof. In another embodiment of the present disclosure, the nanostructure is of spherical shape.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the magnetic material is selected from iron, iron oxide, nickel, nickel oxide, cobalt, cobalt oxide, or combinations thereof. In another embodiment of the present disclosure, the magnetic material is iron or iron oxide.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the core has particle size in a range of 5 to 50 nm. In another embodiment of the present disclosure, the core has particle size in a range of to 45 nm.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the nanostructure has zeta potential in a range of −20 to −40 mV. In another embodiment of the present disclosure, the nanostructure has zeta potential in a range of −25 to −35 mV.

In an embodiment of the present disclosure, there is provided a nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, the nanostructure has zeta potential in a range of −20 to −40 mV, the nanostructure encompass shape selected from sphere, ellipsoids, dumb-bell shaped linked spheres, multiple linked spheres, chains, rods, helices, or combinations thereof, and the nanostructure heals dental hypersensitivity and triggers bone regeneration and growth.

In an embodiment of the present disclosure, there is provided a nanostructure comprising: 50 to 80% (w/w) of iron or iron oxide and 20 to 50% (w/w) of calcium silicate, wherein iron or iron oxide forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the nanostructure heals dental hypersensitivity and triggers bone regeneration and growth.

In an embodiment of the present disclosure, there is provided a nanocomposite comprising: a) 95 to 99.9% (w/w) of the nanostructure comprising (i) 50 to 80% (w/w) of a magnetic material and (ii) 20 to 50% (w/w) of calcium silicate; and b) 0.1 to 5% (w/w) of an additive selected from calcium oxide, phosphorus, sodium, strontium, phosphates, fluorine, or combinations thereof, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm.

In an embodiment of the present disclosure, there is provided a nanocomposite comprising: a) 98 to 99.9% (w/w) of the nanostructure comprising (i) 50 to 80% (w/w) of a magnetic material and (ii) 20 to 50% (w/w) of calcium silicate; and b) 0.1 to 2% (w/w) of an additive selected from calcium oxide, phosphorus, sodium, strontium, phosphates, fluorine, or combinations thereof, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm. In another embodiment of the present disclosure, there is provided the nanocomposite comprising: a) 98.5 to 99.5% (w/w) of the nanostructure comprising (i) 50 to 80% (w/w) of a magnetic material and (ii) 20 to 50% (w/w) of calcium silicate; and b) 0.5 to 1.5% (w/w) of an additive selected from calcium oxide, phosphorus, sodium, strontium, phosphates, fluorine, or combinations thereof, wherein the magnetic particle forms a core of the nanostructure and calcium silicate forms a shell of the nanostructure; the nanostructure has particle size in a range of 100 to 450 nm; surface area in a range of 1 μm² to 100 μm²; and pore size in a range of 4 nm to 18 nm.

In an embodiment of the present disclosure, there is provided a nanocomposite comprising: a) 95 to 99.9% (w/w) of the nanostructure comprising (i) 50 to 80% (w/w) of a magnetic material and (ii) 20 to 50% (w/w) of calcium silicate; and b) 0.1 to 5% (w/w) of an additive selected from calcium oxide, phosphorus, sodium, strontium, phosphates, fluorine, or combinations thereof, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, and the additive is embedded with calcium silicate in the shell of the nanostructure. In another embodiment of the present disclosure, wherein the additive is calcium oxide which is embedded with calcium silicate in the shell of the nanostructure.

In an embodiment of the present disclosure, there is provided a nanocomposite comprising: a) 95 to 99.9% (w/w) of the nanostructure comprising (i) 50 to 80% (w/w) of iron or iron oxide and (ii) 20 to 50% (w/w) of calcium silicate; and b) 0.1 to 5% (w/w) of an additive selected from calcium oxide, phosphorus, sodium, strontium, phosphates, fluorine, or combinations thereof, wherein iron or iron oxide forms a core of the nanostructure; calcium silicate and the additive forms a shell of the nanostructure; the nanostructure has particle size in a range of 50 to 500 nm; surface area in a range of 0.01 μm² to 100 μm²; and pore size in a range of 2 nm to 20 nm.

In an embodiment of the present disclosure, there is provided a process for preparing the nanostructure, the process comprising: (a) mixing an oxidizing agent, and a magnetic metal salt in a first solvent followed by addition of a base and a silicate precursor to obtain a first mixture; (b) contacting a calcium salt with the first mixture in the presence of a second solvent to obtain a second mixture; and (c) annealing the second mixture at a temperature in a range of 550 to 650° C. to obtain a glassy calcium silicate as the shell of the nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm. In another embodiment of the present disclosure, annealing the second mixture is carried out at a temperature in a range of 575 to 625° C. to obtain a glassy calcium silicate as the shell of the nanostructure. In a further embodiment of the present disclosure, the nanostructure has particle size in a range of 100 to 450 nm; surface area in a range of 1 μm² to 100 μm²; and pore size in a range of 4 nm to 18 nm.

In an embodiment of the present disclosure, there is provided a process for preparing the nanostructure as disclosed herein, wherein the oxidizing agent is selected from sodium acetate, sodium citrate, or combinations thereof; the magnetic metal salt is selected from iron chloride, iron nitrate, nickel chloride, nickel nitrate, cobalt chloride, cobalt nitrate, or combinations thereof; the first solvent is selected from ethylene glycol, ethanol, water, hydrochloric acid, or combinations thereof; the base is selected from ammonium hydroxide, or metal hydroxide; and the silicate precursor is selected from tetraethyl orthosilicate (TEOS), tetramethylorthosilicate (TMOS), polyethoxydisiloxane (PEDS), methyltriethoxysilane (MTES) or combinations thereof.

In an embodiment of the present disclosure, there is provided a process for preparing the nanostructure as disclosed herein, wherein the calcium salt is selected from calcium nitrate, calcium carbonate, calcium phosphate, calcium chloride, or combinations thereof; and the second solvent is selected from ethanol, water, or combinations thereof.

In an embodiment of the present disclosure, there is provided a process for preparing the nanostructure as disclosed herein, wherein annealing is carried out for a time period in a range of 2 to 15 hours.

In an embodiment of the present disclosure, there is provided a process for preparing the nanostructure, the process comprising: (a) mixing an oxidizing agent selected from sodium acetate, sodium citrate, or combinations thereof, and a magnetic metal salt selected from iron chloride, iron nitrate, nickel chloride, nickel nitrate, cobalt chloride, cobalt nitrate, or combinations thereof in a first solvent selected from ethylene glycol, ethanol, water, hydrochloric acid, or combinations thereof followed by addition of ammonium hydroxide, or metal hydroxide and a silicate precursor selected from tetraethyl orthosilicate (TEOS), tetramethylorthosilicate (TMOS), polyethoxydisiloxane (PEDS), methyltriethoxysilane (MTES) or combinations thereof, to obtain a first mixture; (b) contacting a calcium salt selected from calcium nitrate, calcium carbonate, calcium phosphate, calcium chloride, or combinations thereof with the first mixture in the presence of a second solvent selected from ethanol, water, or combinations thereof to obtain a second mixture; and (c) annealing the second mixture at a temperature in a range of 550 to 650° C. to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm.

In an embodiment of the present disclosure, there is provided a process for preparing the nanostructure, the process comprising: (a) mixing an oxidizing agent, and a magnetic metal salt in a first solvent followed by addition of a base and a silicate precursor to obtain a first mixture; (b) contacting a calcium salt with the first mixture in the presence of a second solvent to obtain a second mixture; and (c) annealing the second mixture at a temperature in a range of 550 to 650° C. is carried out for a time period in a range of 2 to 15 hours to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm, the annealing process comprises ramping up of temperature for 2 to 5 hours, hold at annealing temperature for 2 to 4 hours, and ramping down of temperature for 2 to 5 hours.

In an embodiment of the present disclosure, there is provided a process for preparing the nanostructure, the process comprising: (a) mixing an oxidizing agent, and a magnetic metal salt in a first solvent followed by heating at a temperature in a range of 200 to 250° C. for a time period of 8 to 12 hours followed by addition of a base and a silicate precursor to obtain a first mixture; (b) contacting a calcium salt with the first mixture in the presence of a second solvent to obtain a second mixture; and (c) annealing the second mixture at a temperature in a range of 550 to 650° C. to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm, In another embodiment of the present disclosure, the mixing of the oxidizing agent, the magnetic metal salt in the first solvent followed by heating at a temperature in a range of 210 to 240° C. for a time period of 9 to 11 hours.

In an embodiment of the present disclosure, there is provided a process for preparing the nanostructure, the process comprising: (a) mixing an oxidizing agent selected from sodium acetate, sodium citrate, or combinations thereof, and a magnetic metal salt selected from iron chloride, iron nitrate, nickel chloride, nickel nitrate, cobalt chloride, cobalt nitrate, or combinations thereof in a first solvent selected from ethylene glycol, ethanol, water, hydrochloric acid, or combinations thereof followed by addition of ammonium hydroxide, or metal hydroxide and a silicate precursor selected from tetraethyl orthosilicate (TEOS), tetramethylorthosilicate (TMOS), polyethoxydisiloxane (PEDS), methyltriethoxysilane (MTES) or combinations thereof, to obtain a first mixture; (b) contacting a calcium salt selected from calcium nitrate, calcium carbonate, calcium phosphate, calcium chloride, or combinations thereof with the first mixture in the presence of a second solvent selected from ethanol, water, or combinations thereof to obtain a second mixture; and (c) annealing the second mixture for a time period in a range of 2 to 15 hours, at a temperature in a range of 550 to 650° C. to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm, and wherein mixing the oxidizing agent, the magnetic metal salt in the first solvent is followed by heating at a temperature in a range of 200 to 250° C. for a time period of 8 to 12 hours, prior to addition of the base and the silicate precursor.

In an embodiment of the present disclosure, there is provided a process of preparing the nanocomposite, the process comprising: a) preparing the nanostructure as disclosed herein; and b) immersing the nanostructure in a solution comprising a compound selected from calcium oxide, strontium salt, calcium fluoride, sodium fluoride, phosphates, or combinations thereof, at a temperature in a range of 20 to 80° C. to obtain the nanocomposite. In another embodiment of the present disclosure, wherein the solution is an aqueous solution comprising a compound selected from calcium oxide, strontium salt, calcium fluoride, sodium fluoride, phosphates, or combinations thereof.

In an embodiment of the present disclosure, there is provided a process of preparing the nanocomposite insitu and on-demand, the process comprising: a) preparing the nanostructure as disclosed herein; and b) immersing the nanostructure in a solution comprising a compound selected from calcium oxide, strontium salt, calcium fluoride, sodium fluoride, phosphates, or combinations thereof, at a temperature in a range of 20 to 80° C. to obtain the nanocomposite.

In an embodiment of the present disclosure, there is provided a nanocomposite wherein the nanostructure with mesoporous nature can absorb the compound selected from calcium oxide, strontium salt, calcium fluoride, sodium fluoride, phosphates, or combinations thereof in trace amounts due to porosity induced increased surface area.

In an embodiment of the present disclosure, there is provided a process of preparing the nanocomposite, the process comprising: a) preparing the nanostructure by the process comprising: (i) mixing an oxidizing agent, and a magnetic metal salt in a first solvent followed by addition of a base and a silicate precursor to obtain a first mixture; (ii) contacting a calcium salt with the first mixture in the presence of a second solvent to obtain a second mixture; and (iii) annealing the second mixture at a temperature in a range of 550 to 650° C. to obtain a glassy calcium silicate as the shell of nanostructure, followed by filtration and drying to obtain the nanostructure, wherein the nanostructure has particle size in a range of 50 to 500 nm; surface area in a range of 0.01 μm² to 100 μm²; and pore size in a range of 2 nm to 20 nm; and b) immersing the nanostructure in a solution comprising a compound selected from calcium oxide, strontium salt, calcium fluoride, sodium fluoride, phosphates, or combinations thereof, at a temperature in a range of 20 to 80° C. to obtain the nanocomposite.

In an embodiment of the present disclosure, there is provided a gel comprising: a) 0.1 to 10% (w/w) of the nanostructure or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; and g) optionally 1 to 5% (w/w) of a stabilizer. In another embodiment of the present disclosure, there is provided a gel comprising: a) 0.1 to 4% (w/w) of the nanostructure or the nanocomposite as disclosed herein; b) 0.1 to 8% (w/w) of a hydrogel base; c) 0.3 to 0.8% (w/w) of a pH modifier; d) 6.5 to 8.5% (w/w) of a humectant; e) 85 to 94% (w/w) of a swelling agent; f) 0.1% to 12% (w/w) of calcium oxide; and f) optionally 1.5 to 4.5% (w/w) of a stabilizer.

In an embodiment of the present disclosure, there is provided a gel comprising: a) 0.1 to 10% (w/w) of the nanostructure or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; g) optionally 1 to 5% (w/w) of a stabilizer; and h) an antibacterial agent.

In an embodiment of the present disclosure, there is provided a gel comprising: a) 0.1 to 10% (w/w) of the nanostructure comprising: 50 to 80% (w/w) of a magnetic material and 20 to 50% (w/w) of calcium silicate, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm, or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide and g) optionally 1 to 5% (w/w) of a stabilizer.

In an embodiment of the present disclosure, there is provided a gel comprising: a) 0.1 to 10% (w/w) of the nanostructure as disclosed herein or the nanocomposite comprising: I) 95 to 99.9% (w/w) of the nanostructure comprising (i) 50 to 80% (w/w) of a magnetic material and (ii) 20 to 50% (w/w) of calcium silicate; and II) 0.1 to 5% (w/w) of an additive selected from phosphorus, sodium, strontium, phosphates, fluorine, or combinations thereof, wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; and g) optionally 1 to 5% (w/w) of a stabilizer.

In an embodiment of the present disclosure, there is provided a gel comprising: a) 0.1 to 10% (w/w) of the nanostructure or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; and g) optionally 1 to 5% (w/w) of a stabilizer, wherein the hydrogel base is selected from polyacrylic acid, diutan gum, alkyl acrylate cross polymer, poloxamers compounds, or combinations thereof; the pH modifier is selected from triethanolamine, di-sodium tetraborate, TrisBase, or combinations thereof; the humectant is selected from glycerin, lecithin, propylene glycol, water or combinations thereof; the swelling agent is selected from ethanol, water, or combinations thereof; and the stabilizer is selected from xanthan gum, gelatin, starch, agar glycerides, or combinations thereof.

In an embodiment of the present disclosure, there is provided a gel comprising: a) 0.1 to 10% (w/w) of the nanostructure or the nanocomposite as disclosed herein; b) 0.1 to 20% (w/w) of a hydrogel base; c) 0.1 to 1% (w/w) of a pH modifier; d) 5 to 10% (w/w) of a humectant; e) 80 to 99% (w/w) of a swelling agent; f) 0.1% to 15% (w/w) of calcium oxide; and g) optionally 1 to 5% (w/w) of a stabilizer, wherein the gel has viscosity in a range of 1 to 10⁵ cP and storage modulus in a range of 1 to 1000 Pa. In another embodiment of the present disclosure, the gel has viscosity in a range of 1 to 500 cP and storage modulus in a range of 1 to 500 Pa.

In an embodiment of the present disclosure, there is provided a process for preparing the gel, the process comprising: a) mixing the nanostructure or the nanocomposite as disclosed herein with a hydrogel base with a swelling agent to obtain a first solution; b) adding a pH modifier and calcium oxide powder to the first solution to obtain a second solution; and c) adding a humectant and optionally a stabilizer to obtain the gel.

In an embodiment of the present disclosure, there is provided a process for preparing the gel, the process comprising: a) mixing the nanostructure or the nanocomposite as disclosed herein with a hydrogel base selected from polyacrylic acid, diutan gum, alkyl acrylate cross polymer, poloxamers compounds, or combinations thereof, with a swelling agent selected from ethanol, water, or combinations thereof to obtain a first solution; b) adding a pH modifier selected from triethanolamine, di-sodium tetraborate, TrisBase, or combinations thereof, and calcium oxide powder to the first solution to obtain a second solution; and c) adding a stabilizer selected from xanthan gum, gelatin, starch, agar glycerides, or combinations thereof, a humectant selected from glycerin, lecithin, propylene glycol, or combinations thereof, to obtain the gel.

In an embodiment of the present disclosure, there is provided a method of treating dental hypersensitivity, the method comprising administering the nanostructure or the nanocomposite or the gel as disclosed herein to a subject suffering from dental hypersensitivity.

In an embodiment of the present disclosure, there is provided a method of administering the nanostructure or the nanocomposite or the gel as disclosed herein, the method comprising applying, driving, and positioning the nanostructure or the nanocomposite or the gel as disclosed herein to an infected dentinal tubule using a magnetic cap. In another embodiment of the present disclosure, wherein the method comprising applying, driving, and positioning the nanostructure or the nanocomposite or the gel as disclosed herein to an infected dentinal tubule using a magnetic clip.

In an embodiment of the present disclosure, there is provided a device comprising the nanostructure or the nanocomposite or the gel as disclosed herein and a magnetic cap.

In an embodiment of the present disclosure, there is provided a device comprising the nanostructure or the nanocomposite or the gel as disclosed herein and a magnetic cap, wherein the device further comprises a piezo generator to induce acoustic excitation. In another embodiment of the present disclosure, wherein the magnetic cap or clip along with a piezo generator generates acoustic vibrations to induce shear thinning of the nanostructure or the nanocomposite or the gel.

In an embodiment of the present disclosure, there is provided a device comprising the nanostructure or the nanocomposite or the gel as disclosed herein and a magnetic cap, wherein the magnetic cap or clip comprises a simple permanent magnet or an electromagnetic coil.

In an embodiment of the present disclosure, there is provided a use of the nanostructure or the nanocomposite or the gel as disclosed herein.

In an embodiment of the present disclosure, there is provided the nanostructure or the nanocomposite or the gel as disclosed herein for use in treating dental hypersensitivity and triggering bone regeneration and growth.

Although the subject matter has been described in considerable detail with reference to certain embodiments thereof, other embodiments are possible. As such, the spirit and scope of the disclosure should not be limited to the description of the embodiments contained herein.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

### Example 1

### Nanostructures

For the purpose of long-term prevention or inhibition of DH, the present disclosure discloses the nanostructures which restrict the passage of dentinal tubules, thereby facilitating the disruption of the transmission of nerve impulses from the exposed dentine surfaces to the pulp leading to pain. The nanostructures of the present disclosure have elemental composition in the form of core-shell structures wherein the core is made magnetic by the presence of magnetic materials. The magnetic core allows targeted delivery of the particles into the dentine tubes using an external magnetic force. The nanostructures as disclosed herein result in the prevention or healing DH by triggering bone regeneration and growth.

The core of the nanostructures is made up of magnetic materials like iron, iron oxide, nickel, cobalt, or combinations thereof, having a weight percentage of 50-80%; and the shell is made up of calcium silicate having a weight percentage of 20% to 50%. The nanostructures have particle size in a range of 50 to 500 nm (diameter); surface area in a range of 0.01 μm² to 100 μm²; and pore size in a range of 2 nm to 20 nm. The surface charge (zeta potential) of the nanostructures would lie between −20 to −40 mV, which rendered highly negative and provided stability to them against agglomeration. The negative charge prevented magnetic agglomeration due to inherent similar-charge based repulsion and superparamagnetic behaviour allowed magnetic agglomeration, if any, to be reversed on the removal of the magnetic fields.

Although, the nanostructure as disclosed herein has a spherical shape with an inner core and outer shell structure, it could be contemplated that such a design or composition could be in different shapes like ellipsoids, dumb-bell shaped linked spheres, multiple linked spheres, chains, rods, helices, or combinations thereof. Furthermore, the present disclosure also uses magnetic material selected from the group comprising iron, iron oxide, nickel, nickel oxide, cobalt, cobalt oxide, or combinations thereof to arrive at a varying form of nanostructures as disclosed herein. All such possible forms fall under the ambit of disclosure made herein. For instance, the nanostructures as disclosed herein could also be developed in the form of cookie-like structure, wherein the magnetic core particles are embedded in a calcium silicate matrix.

### Example 2

### Preparation of Nanostructures

For the purpose of the present disclosure, the nanostructures were developed using magnetite (iron oxide, Fe₃O₄) nanocrystals. Initially, 20 ml of ethylene glycol (first solvent) was taken in Teflon tube and 1.2 gm of sodium acetate (oxidizing agent) was added to it followed by the addition of 0.2 gm of sodium citrate dihydrate (oxidizing agent). Sequentially, 1.080 gm of iron chloride hexahydrate (magnetic metal (iron) salt provides 69.9% of iron) was added to the ethylene glycol solution and then the Teflon tube was placed in the autoclave chamber and placed in the hot air oven at 210° C. for 10 hours.

Post completion of 10 hours in the hot air oven, the sample in the Teflon tube was cleaned using centrifugation at 5000 rpm for 5 minutes. The centrifuged sample was then suspended in water and was subjected to subsequent centrifugation three times in water, followed by centrifugation three times in ethanol. After centrifugation steps, the sample was then suspended in 50 ml ethanol, out of which 10 ml of the sample was taken and centrifuged at 5000 rpm for 5 minutes. The centrifuged sample was then suspended in 10 ml of deionized (DI) water. In the meantime, 0.425 ml of 38% HCl was added to 40 ml DI water to prepare HCl solution of 0.1 M (first solvent), which was then added to the 10 ml of the centrifuged sample of the last step. The mixture was then sonicated for a duration of 10 minutes followed by two times centrifugation at 5000 rpm for 5 minutes and then suspended in 10 ml ethanol solution, out of which 5 ml of sample was added to 37.5 ml of ethanol which was then mixed with 3.5 ml of ammonium hydroxide (base) measured with small measuring jar due to low viscosity. The mixture of the last step (i.e., base+first solvent) was then added to 47.5 ml of centrifuged sample (solution of the oxidizing agent, and the magnetic metal salt in a first solvent suspended in 50 ml ethanol above) and then placed in a sonicator bath followed by addition of 0.1 ml tetraethyl orthosilicate (TEOS) solution (silicate precursor) to obtain a first mixture. Post addition of TEOS solution, the first mixture was sonicated for a duration of 90 minutes. During sonication, ice cubes were added after every 5 minutes to maintain the initial temperature. After sonication, the sample was centrifuged in water three times followed by sonication in ethanol three times. (second solvent).

The first mixture was then suspended in 10 ml of ethanol and 5 ml of that solution was taken in a beaker and mixed with 0.13 gm of calcium nitrate tetrahydrate (calcium salt provides 46.5% of calcium silicate) to obtain a second mixture. The second mixture was then sonicated for 55 hours, and ice cubes were added after every 5 minutes to maintain the room temperature. Post sonication, the sample was placed on a magnet to remove an excess amount of calcium nitrate. After magnetic separation, 2.5 ml of ethanol (second solvent) was added to the magnetically separated sample and placed in the oven at 40° C. under forced air condition till the ethanol solution evaporated. After complete evaporation of ethanol, the obtained second mixture was placed in Lindberg furnace and subjected to annealing at 600° C. for 3 hours. The temperature was serially ramped at the rate of 2° C. per minutes (5 hours). The sample was taken out from the furnace after completion of cycle and powder was scrapped from quartz crucible. The sample was then suspended in water and centrifuged thrice to obtain the nanostructure.

In the process as elaborated above, the magnetite nanocrystals were prepared followed by the formation of a glassy calcium silicate as the shell of the nanostructure. Calcium was infused into the iron-silicate core shell using calcium nitrate tetrahydrate.

It was observed that increasing the weight percentage of calcium silicate resulted in the formation of calcium nodules and eventually agglomeration of nanostructures. Thus, increasing the weight percentage of calcium silicate in the nanostructure beyond 20-50% was not possible without substantial changes to the geometry and size. Accordingly, the nanostructures having more than 50% of calcium silicate in the composition with similar geometry and size as disclosed herein did not show the desired properties as exhibited by the nanostructures of the present disclosure.

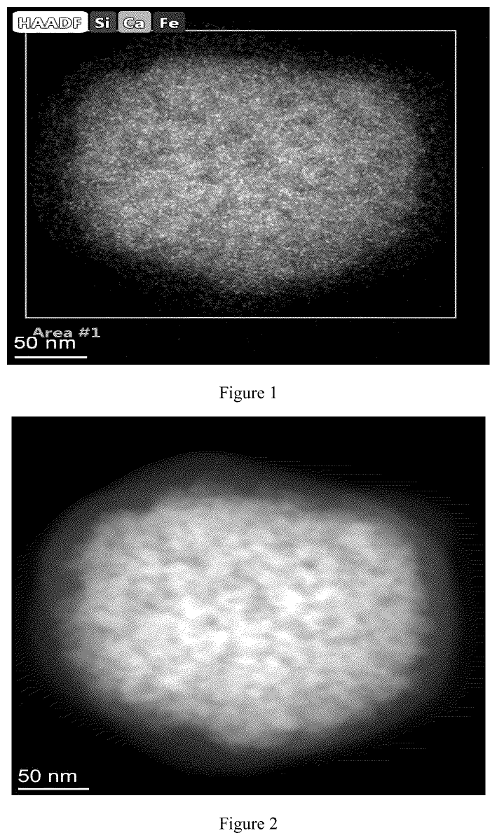

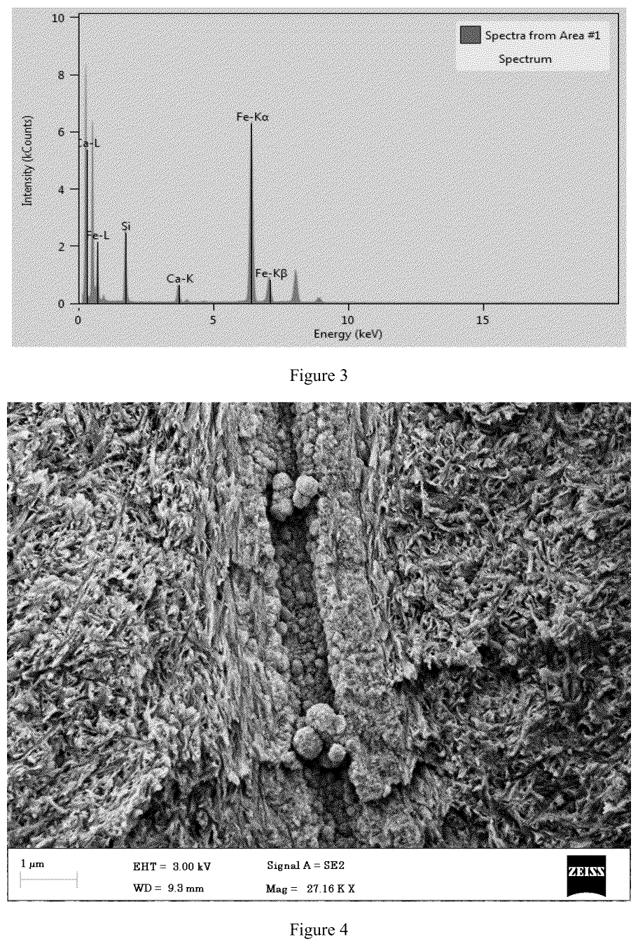

Overall, it could be inferred that the particle size in a range of 50 to 500 nm; surface area in a range of 0.01 μm² to 100 μm²; and pore size in a range of 2 nm to 20 nm, are the critical aspects of the nanostructures as disclosed herein. High-angle annular dark-field imaging (HAADF) of scanning transmission electron microscopic analysis indicated that the particle size was 250 nm (FIGS. 1 and 2) with the pore size was about 10 nm and surface area in a range of 1 to 100 μm². Energy-dispersive x-ray spectroscopic analysis (FIG. 3) further confirmed the presence of magnetic particle (iron) and calcium silicate particles in the nanostructure.

Further, the varying forms of nanostructures as disclosed herein, comprised 50 to 80% (w/w) of a magnetic material and 20 to 50% (w/w) of calcium silicate, wherein the magnetic particle formed a core of the nanostructure and calcium silicate formed a shell of the nanostructure.

### Example 3

### Nanostructure Based Nanocomposite

In accordance with the disclosure made herein, the nanocomposite comprises 95 to 99.9% (w/w) of the nanostructure (prepared in Example 2); and 0.1 to 5% (w/w) of an additive selected from calcium oxide, phosphorus, sodium, strontium, phosphates, fluorine, or combinations thereof. In specific the nanocomposite was obtained by adding nanostructure as obtained in Example 2 with calcium oxide dissolved in water. The addition of such additives allowed the formation of nanocomposite as needed on demand inside microscopic channels such as dentinal tubules.

### Example 4

### Preparation of Nanocomposite

The process of preparing the nanocomposite as disclosed herein comprises a) preparing the nanostructure (as prepared in Example 2 hereinabove); and b) immersing the nanostructure in a calcium oxide solution. The process of physisorption was expedited by using increased ambient temperature conditions, in a range of 20 to 80° C. Calcium oxide solution reacted with the nanostructure to form cement like materials in-situ. The addition of such additives allowed the formation of nanocomposite as needed on demand inside microscopic channels such as dentinal tubules.

Although calcium oxide had been used in the present example for physisorption in the nanostructures, additives could be selected from strontium salts, sodium fluoride, calcium fluoride, phosphates, fluorides, or combinations thereof using various surface absorption methods in place of physisorption for similar purposes can also be prepared.

### Example 5

### Nanostructure Based gel Composition

The present example relates to a gel composition comprising the nanostructures or the nanocomposite and a process for the preparation of topical gel for use in dental healthcare for the prevention or treatment of DH. The present disclosure provide a carbomer-triethanolamine based gel with trace amounts of strontium and fluoride salts to help desensitize the dentinal pain. The gel composition comprised, 50 ml of distilled water (swelling agent), 5 mg of calcium oxide powder, 0.01% (w/w) of 3% hydrogen peroxide (antibacterial agent), 5.2% (w/w) anhydrous glycerine (humectant), 0.2% (w/w) carbomer 980 or other carbomer compounds (hydrogel base), 0.15 ml (0.3% (w/w)) triethanolamine (pH modifier), 0.5% (w/w) calcium oxide, and 2% (w/w) of nanocomposite comprising 99.5% (w/w) of the nanostructure (prepared in Example 2) with 0.5% (w/w) of an additive selected from calcium oxide (0.1 mg/ml), calcium fluoride or strontium chloride (additives) each. The gel as disclosed herein had a viscosity of 5000 cP and a storage modulus of 38.3 Pa (at a shear rate of 1 rad/sec).

### Example 6

### Preparation of Nanostructure Based gel Composition

The present example relates to the method of preparation of nanostructure-based gel composition as disclosed in Example 5. The process involved the steps of mixing 2% (w/w) of nanostructure as prepared in Example 2 with 0.2% (w/w) of hydrogel base followed by subsequent mixing with 50 ml of distilled water (swelling agent) to obtain a first solution. To the first solution, 5 mg of calcium oxide powder, and 0.15 ml (0.3% (w/w)) of triethanolamine (pH modifier) were added in order to obtain a second solution. After the preparation of second solution, 5.2% (w/w) of anhydrous glycerin (humectant) was added to obtain the gel. In addition, 0.01% (w/w) of 3% hydrogen peroxide was added to the gel in order to impart antibacterial properties.

### Example 7

### Nanostructure Based Device

In order to facilitate the applications of the nanostructure or nanocomposite or gel composition as disclosed herein, the present disclosure provides a device comprising the nanostructure or the nanocomposite or the gel and a magnetic cap. The device as disclosed herein further comprises a piezo generator to induce acoustic excitation.

In accordance with the present disclosure, the magnetic cap or clip comprises a simple permanent magnet or an electromagnetic coil which regulates the targeted motion of nanostructures under the magnetic field. For the purpose of treatment or prevention of DH in a subject, the gel composition is applied over the hypersensitive spots (exposed dentins) and the magnetic cap or clip is placed on the other side of the affected region in tooth/dental arch (Maxilla/Mandible). The magnetic cap or clip pulls the nanostructures present in the gel suspension and directs them towards the exposed dentinal tubules for 10-15 minutes during which the subject's head is stabilized in a neutral rest position for improving the efficacy of the treatment.

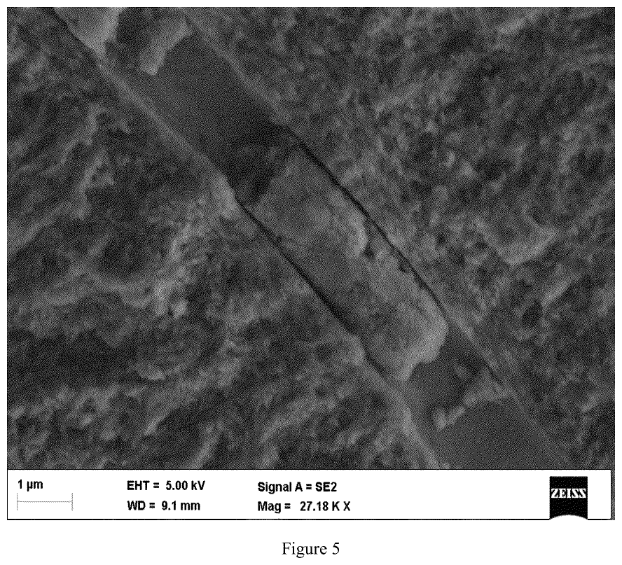

The nanostructures on applying on affected tooth, navigate into the dentinal tubules and form blockages in between to suspend the movement of nerve impulses as shown in FIG. 4. After the treatment session the device is removed, and the remnant gel is wiped off from the subject's tooth. The subject might have to take subsequent sessions for improving the efficacy of the treatment. FIG. 4 shows microscopic view capture depicting evident tubular blockage via nanostructures at two different places. Similarly the nanostructure comprising iron with calcium silicate, was mixed with calcium oxide solution and applied on the affected tooth to form a nanocomposite in-situ on demand. FIG. 5 depict the microscopic image of the nanocomposite formed, which indicated the formation of cement block on the dentinal tubules. This confirmed ability of the nanocomposite of the present disclosure to form cementing blocks on the passage, thereby forming permanent blockage of the dentinal tubules, preventing hypersensitivity and associated pain.

### Advantages of the Present Disclosure

The above-mentioned implementation examples as described on this subject matter and its equivalent thereof have many advantages, including those which are described.

The present disclosure discloses a nanostructure, a nanocomposite, a gel composition, an oral healthcare device, and process of preparing thereof respectively. Unlike conventional medications which get cleared off from the site of sensitivity after a period of time, the nanostructures as disclosed herein navigate into the dentinal tubules and block the passage, thereby restricting the nerve impulse signals from the exposed dentins to the pulp resulting in a lack of DH/pain. Furthermore, the calcium in the nanostructures is supposed to trigger bone growth in these exposed dentines thus forming a more permanent blockage. The strontium doping would desensitize the nerve ending thereby substantially reducing pain.

The nanostructures as disclosed herein also have the regenerative capabilities causing repair of the mineralized tissues through triggering regenerative cell signaling processes. Along with their regenerative ability, they can be remotely homed towards the affected areas through generating directive electromagnetic fields from a hand-held device due to their innate magnetic nature. With these attributes, said nanostructures can be purposed for regeneration and repair of mineralized tissues such as bones, ligaments, and teeth.

1. A nanostructure comprising:
50 to 80% (w/w) of a magnetic material; and 20 to 50% (w/w) of calcium silicate, wherein the magnetic particle forms a core of the nanostructure and calcium silicate forms a shell of the nanostructure;
the nanostructure has particle size in a range of 50 to 500 nm; surface area in a range of 0.01 μm² to 100 μm²; and pore size in a range of 2 nm to 20 nm.

2. The nanostructure as claimed in claim 1, wherein the nanostructure encompass shape selected from sphere, ellipsoids, dumb-bell shaped linked spheres, multiple linked spheres, chains, rods, helices, or combinations thereof.

3. The nanostructure as claimed in claim 1, wherein the magnetic material is selected from iron, iron oxide, nickel, nickel oxide, cobalt, cobalt oxide, or combinations thereof.

4. The nanostructure as claimed in claim 1, wherein the core has particle size in a range of 5 to 400 nm.

5. (canceled)

6. The nanostructure as claimed in claim 1, wherein the nanostructure has zeta potential in a range of −20 to −40 mV; and the nanostructure heals dental hypersensitivity and triggers bone regeneration and growth.

7. A nanocomposite comprising:
95 to 99.9% (w/w) of the nanostructure comprising (i) 50 to 80% (w/w) of a magnetic material and (ii) 20 to 50% (w/w) of calcium silicate; and
b) 0.1 to 5% (w/w) of an additive selected from calcium oxide, phosphorus, sodium, strontium, phosphates, fluorine, or combinations thereof,
wherein the magnetic particle forms a core of the nanostructure and calcium silicate forms a shell of the nanostructure; the nanostructure has particle size in a range of 50 to 500 nm; surface area in a range of 0.01 μm² to 100 μm²; and pore size in a range of 2 nm to 20 nm.

8. The nanocomposite as claimed in claim 7, wherein the additive is embedded with calcium silicate in the shell of the nanostructure; and calcium oxide is embedded with calcium silicate in the shell of the nanostructure.

9. (canceled)

10. A process for preparing the nanostructure as claimed in claim 1, the process comprising:
mixing an oxidizing agent, and a magnetic metal salt in a first solvent followed by addition of a base and a silicate precursor to obtain a first mixture;
contacting a calcium salt with the first mixture in the presence of a second solvent to obtain a second mixture; and
annealing the second mixture at a temperature in a range of 550 to 650° C. to obtain a glassy calcium silicate as the shell of the nanostructure, followed by filtration and drying to obtain the nanostructure,
wherein the nanostructure has particle size in a range of 50 to 500 nm; surface area in a range of 0.01 μm² to 100 μm²; and pore size in a range of 2 nm to 20 nm.

11. The process as claimed in claim 10, wherein the oxidizing agent is selected from sodium acetate, sodium citrate, or combinations thereof; the magnetic metal salt is selected from iron chloride, iron nitrate, nickel chloride, nickel nitrate, cobalt chloride, cobalt nitrate, or combinations thereof; the first solvent is selected from ethylene glycol, ethanol, water, hydrochloric acid, or combinations thereof; the base is selected from ammonium hydroxide, or metal hydroxide; and the silicate precursor is selected from tetraethyl orthosilicate (TEOS), tetramethylorthosilicate (TMOS), polyethoxydisiloxane (PEDS), methyltriethoxysilane (MTES) or combinations thereof; the calcium salt is selected from calcium nitrate, calcium carbonate, calcium phosphate, calcium chloride, or combinations thereof; and the second solvent is selected from ethanol, water, or combinations thereof.

12. (canceled)

13. The process as claimed in claim 10, wherein annealing is carried out for a time period in a range of 2 to 15 hours; and mixing the oxidizing agent, the magnetic metal salt in the first solvent is followed by heating at a temperature in a range of 200 to 250° C. for a time period of 8 to 12 hours, prior to addition of the base and the silicate precursor.

14. (canceled)

15. A process of preparing a nanocomposite, the process comprising: a) preparing the nanostructure as claimed in claim 10; and b) immersing the nanostructure in a solution comprising a compound selected from calcium oxide, strontium salt, calcium fluoride, sodium fluoride, phosphates, or combinations thereof, at a temperature in a range of 20 to 80° C. to obtain the nanocomposite.

16. A gel comprising:
a. 0.1 to 10% (w/w) of the nanocomposite as claimed in claims 7;
b. 0.1 to 20% (w/w) of a hydrogel base;
c. 0.1 to 1% (w/w) of a pH modifier;
d. 5 to 10% (w/w) of a humectant;
e. 80 to 99% (w/w) of a swelling agent;
f. 0.1% to 15% (w/w) of calcium oxide; and
g. optionally 1 to 5% (w/w) of a stabilizer.

17. The gel as claimed in claim 16 further comprises an antibacterial agent.

18. The gel as claimed in claim 16, wherein the hydrogel base is selected from polyacrylic acid, diutan gum, alkyl acrylate cross polymer, poloxamers compounds, or combinations thereof; the pH modifier is selected from triethanolamine, di-sodium tetraborate, TrisBase, or combinations thereof; the humectant is selected from glycerin, lecithin, propylene glycol, or combinations thereof; the swelling agent is selected from ethanol, water, or combinations thereof; and the stabilizer is selected from xanthan gum, gelatin, starch, agar glycerides, or combinations thereof.

19. The gel as claimed in claim 16, wherein the gel has viscosity in a range of 1 to 105 cP; and storage modulus in a range of 1 to 1000 Pa.

20. A process for preparing the gel as claimed in claim 16, the process comprising:
a. mixing the nanocomposite, with a hydrogel base and a swelling agent to obtain a first solution;
b. adding a pH modifier and calcium oxide powder to the first solution to obtain a second solution; and
c. adding a humectant and optionally a stabilizer, to obtain the gel.

21. A method of treating dental hypersensitivity, the method comprising administering the nanostructure as claimed in claim 1 to a subject suffering from dental hypersensitivity.

22. A method of administering the nanostructure as claimed in claim 1, the method comprising applying, driving, and positioning the nanostructure as claimed in claim 1 to an infected dentinal tubule using a magnetic cap; and wherein the magnetic cap comprises a simple permanent magnet or an electromagnetic coil.

23. A device comprising the nanostructure as claimed in claim 1 and a magnetic cap; and
the magnetic cap comprises a simple permanent magnet or an electromagnetic coil.

24. The device as claimed in claim 23, wherein the device further comprises a piezo generator to induce acoustic excitation.

25.-26. (canceled)
